(19) United States
(12) Patent Application Publication
(10) Pub. No.: US 2026/0317706 A1
(43) Pub. Date: Oct. 15, 2026

(54) COMPOSITION FOR INDUCING STEM CELLS TO DIFFERENTIATE INTO ASTROCYTES, CONTAINING CPI-455

(71) Applicant: CHUNG ANG UNIVERSITY INDUSTRY ACADEMIC COOPERATION FOUNDATION, Seoul (KR)

(72) Inventor: Hyun Jung KIM, Seoul (KR)

(21) Appl. No.: 19/472,177

(22) PCT Filed: Apr. 4, 2024
(86) PCT No.: PCT/KR2024/004453

§ 371 Date: Oct. 3, 2025

(30) Foreign Application Priority Data
Apr. 5, 2023 (KR) 10-2023-0044632

(51) Int. Cl. C12N 5/079; A61K 31/519; A61K 35/30

(52) U.S. Cl. CPC C12N 5/0622; A61K 31/519; A61K 35/30; C12N 2501/155; C12N 2501/60; C12N 2506/08

(57) ABSTRACT

The present invention relates to a composition for inducing stem cells to differentiate into astrocytes, containing CPI-455. By using the composition of the present invention, differentiation into astrocytes can be induced without using a conventional stem cell differentiation promoter, and it is also expected that the differentiated astrocytes can be used to effectively treat neurodegenerative diseases.

### TECHNICAL FIELD

The present invention relates to a composition for inducing stem cells to differentiate into astrocytes, comprising CPI-455.

This application claims priority to and the benefit of Korean Patent Application No. 10-2023-0044632, filed on Apr. 5, 2023, the disclosure of which is incorporated herein by reference in its entirety.

### BACKGROUND ART

The term “neurodegenerative disorders” refers to disorders that affect the brain or spinal cord among degenerative disorders that occur as we age, and to disorders that are manifested by the gradual loss of structure and function of specific brain cell populations in the brain and spinal cord due to as yet unknown causes or to genetic defects or environmental factors. It is known that neuronal cell death and damage caused by degenerative nervous system diseases cause problems in the formation or function of synapses that transmit information between neurons, which is most important for transmitting information in the brain nervous system as well as abnormal increases or decreases in electrical activity of brain neurons. Since neurons in the brain and spinal cord perform a wide variety of functions depending on their location, damage to neuronal cells in a specific area causes a characteristic dysfunction, and depending on how this dysfunction progresses, a wide variety of clinical manifestations are also seen. Such degenerative nervous system diseases include amyotrophic lateral sclerosis (ALS), also known as Lou Gehrig's disease, Parkinson's disease (PD), Alzheimer's disease (AD), Huntington's disease (HD), multiple sclerosis (MS), and the like.

Meanwhile, to date, for research on Alzheimer's disease, research has mainly focused on the pathogenesis of the disease and has been conducted by inducing cells to differentiate into neurons, which are abundant in the forebrain. Differentiation into forebrain neurons is carried out by a neuron differentiation method that proceeds without the addition of any specific external substance. Recently, a monolayer culture method through ‘dual-SMAD inhibition’, a method through embryoid body formation, a method through cerebral organoid formation and the like have been developed, and both ‘glutamatergic neurons’ and ‘GABAergic neurons’ are widely used in research on the discovery of the pathogenesis of Alzheimer's disease. According to a recently published paper, when patient-derived induced pluripotent stem cells were differentiated into “GABAergic neurons,” it was confirmed that they were more resistant to cell death caused by amyloid beta. In other words, this shows that differentiation into specific neurons may result in various phenotypes.

Along with neurons, astrocytes have also been reported to play a crucial role in the pathogenesis of Alzheimer's disease. It has been reported that astrocytes play a critical role in synapse formation and maturation and thus can treat neurodegenerative disorders. In general, astrocytes differentiate from the same precursor cells as neurons, and it is possible to fractionate and purify the cells from a cell population that contains both types of cells using astrocyte-specific expression factors.

Meanwhile, it is not known whether CPI-455 is effective in inducing neural stem cells into astrocytes.

### DETAILED DESCRIPTION OF INVENTION

### Technical Problem

The inventors of the present invention, as a result of diligent efforts to find a method for inducing stem cells into astrocytes, completed the present invention by confirming that CPI-455 of the present invention may be treated to induce neural stem cells into astrocytes.

Accordingly, an object of the present invention is to provide a composition for inducing stem cells to differentiate into astrocytes, comprising CPI-455 as an active ingredient.

Another object of the present invention is to provide a method for differentiating stem cells into astrocytes, the method comprising treating the stem cells with the composition for inducing stem cells to differentiate into astrocytes according to the present invention and culturing the stem cells.

Another object of the present invention is to provide a kit for inducing stem cells to differentiate into astrocytes, comprising CPI-455 or the composition for inducing stem cells to differentiate into astrocytes according to the present invention.

Another object of the present invention is to provide a use of CPI-455 or the composition for inducing stem cells to differentiate into astrocytes according to the present invention for inducing stem cells to differentiate into astrocytes.

Yet another object of the present invention is to provide a pharmaceutical composition for preventing or treating a neurodegenerative disorder, comprising CPI-455 as an active ingredient.

Yet another object of the present invention is to provide a method for preventing or treating a neurodegenerative disorder, the method comprising administering CPI-455, or a composition comprising CPI-455 as an active ingredient, to a subject in need thereof.

Yet another object of the present invention is to provide a use of CPI-455, or a composition comprising CPI-455 as an active ingredient, for preventing or treating a neurodegenerative disorder.

Yet another object of the present invention is to provide a use of CPI-455, or a composition comprising CPI-455 as an active ingredient, for preparing a therapeutic agent for neurodegenerative disorders.

Yet another object of the present invention is to provide a cell therapy agent for preventing or treating a neurodegenerative disorder, comprising astrocytes prepared by the method of differentiating stem cells into astrocytes according to the present invention.

Yet another object of the present invention is to provide a method for preventing or treating a neurodegenerative disorder, the method comprising administering the cell therapy agent according to the present invention to a subject in need thereof.

Yet another object of the present invention is to provide a use of the cell therapy agent according to the present invention for preventing or treating a neurodegenerative disorder.

Yet another object of the present invention is to provide a use of the astrocytes prepared by the method of differentiating stem cells into astrocytes according to the present invention for preparing a cell therapy agent.

However, technical problems to be solved in the present invention are not limited to the above-described problems, and other problems which are not described herein will be fully understood by those of ordinary skill in the art from the following descriptions.

### Technical Solution

To achieve the above object, the present invention provides a composition for inducing stem cells to differentiate into astrocytes, comprising CPI-455 as an active ingredient.

In one embodiment of the present invention, the stem cells may be one or more selected from the group consisting of embryonic stem cells, adult stem cells, embryonic germ stem cells, and embryonic tumor stem cells, but are not limited thereto.

In another embodiment of the present invention, the CPI-455 may be included at a concentration of 0.1 μM to 100 μM based on the total composition, but is not limited thereto.

In yet another embodiment of the present invention, the CPI-455 may satisfy one or more of the following characteristics, but is not limited thereto:
(a) promoting phosphorylation of signal transducer and activator of transcription 3 (STAT3); and
(b) increasing secretion of bone morphogenic protein 2 (BMP2).

Additionally, the present invention provides a method for differentiating stem cells into astrocytes, the method comprising treating the stem cells with the composition for inducing stem cells to differentiate into astrocytes according to the present invention and culturing the stem cells.

In one embodiment of the present invention, the astrocytes may express glial fibrillary acidic protein (GFAP), but are not limited thereto.

Additionally, the present invention provides a kit for inducing stem cells to differentiate into astrocytes, comprising CPI-455 or the composition for inducing stem cells to differentiate into astrocytes according to the present invention.

Additionally, the present invention provides a use of CPI-455 or the composition for inducing stem cells to differentiate into astrocytes according to the present invention for inducing stem cells to differentiate into astrocytes.

Additionally, the present invention provides a pharmaceutical composition for preventing or treating a neurodegenerative disorder, comprising CPI-455 as an active ingredient.

Additionally, the present invention provides a cell therapy agent for preventing or treating a neurodegenerative disorder, comprising astrocytes prepared by the method of differentiating stem cells into astrocytes according to the present invention.

In one embodiment of the present invention, the neurodegenerative disorder may be one or more selected from the group consisting of Alzheimer's disease, amyotrophic lateral sclerosis, adrenoleukodystrophy, Alexander's disease, Alper's disease, ataxia telangiectasia, Batten's disease, bovine spongiform encephalopathy (BSE), Canavan's disease, corticobasal degeneration, Creutzfeldt-Jakob disease, dementia with Lewy bodies, fatal familial insomnia, frontotemporal lobar degeneration, Huntington's disease, Kennedy's disease, Krabbe's disease, Lyme disease, Machado-Joseph disease, multiple sclerosis, multiple system atrophy, neuroacathocytosis, Niemann-Pick's disease, Parkinson's disease, Pick's disease, primary lateral sclerosis, progressive supranuclear palsy, Refsum's disease, Sandhoff's disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome, but is not limited thereto.

Additionally, the present invention provides a method for preventing or treating a neurodegenerative disorder, the method comprising administering CPI-455, or a composition comprising CPI-455 as an active ingredient, to a subject in need thereof.

Additionally, the present invention provides a use of CPI-455, or a composition comprising CPI-455 as an active ingredient, for preventing or treating a neurodegenerative disorder.

Additionally, the present invention provides a use of CPI-455, or a composition comprising CPI-455 as an active ingredient, for preparing a therapeutic agent for neurodegenerative disorders.

Additionally, the present invention provides a method for preventing or treating a neurodegenerative disorder, the method comprising administering the cell therapy agent according to the present invention to a subject in need thereof.

Additionally, the present invention provides a use of the cell therapy agent according to the present invention for preventing or treating a neurodegenerative disorder.

Additionally, the present invention provides a use of the astrocytes prepared by the method of differentiating stem cells into astrocytes according to the present invention for preparing a cell therapy agent.

### Advantageous Effects

The inventors of the present invention, by exhibiting a technique for inducing stem cells to differentiate into astrocytes using CPI-455, expect not only to be able to induce differentiation into astrocytes without the use of existing stem cell differentiation promoters, but also to be able to effectively treat neurodegenerative disorders using the differentiated astrocytes.

### DESCRIPTION OF DRAWINGS

FIG. 1A shows the results of western blot analysis of the expression levels of TUBB3 (Tubulin beta 3 class III), GFAP, and OLIG2 (Oligodendrocyte transcription factor 2) after treating neural stem cells with 0.1% DMSO or CPI-455 for 4 days without EGF and FGF2 (D: DMSO, same below).

FIG. 1B shows the results of RT-qPCR measurement of tubb3 and gfap mRNA expression after treating neural stem cells with 0.1% DMSO or CPI-455 for 3 days without EGF and FGF2.

FIG. 2A shows the results of immunostaining with TUBB3 (green) or anti-GFAP (red) after treating neural stem cells with 0.1% DMSO or CPI-455 for 4 days without EGF and FGF2. (Top) 0.1% DMSO treatment, (Bottom) CPI-455 treatment. Nuclei were stained with DAPI (blue). Scale bar=100 μm.

FIG. 2B shows the ratio of TUBB3-positive cells and GFAP-positive cells to DAPI-positive cells after treating neural stem cells with 0.1% DMSO or CPI-455 for 4 days without EGF and FGF2.

FIG. 3A shows the results of western blot analysis of STAT3 phosphorylation levels in neural stem cells treated with CPI-455.

FIG. 3B shows the results of RT-qPCR measurement of il-6, lif, cntf, and bmp2 mRNA expression in neural stem cells treated with CPI-455.

### BEST MODE

The terms, technologies, and the like used in the present specification, unless otherwise defined, are used in the meanings generally employed in the technical field to which the present invention pertains. In addition, all documents referred to in the present specification are incorporated herein as references for explaining the present invention.

In the present specification, the term “active ingredient” refers to a component that exhibits the intended activity alone, or exhibits the intended activity in combination with a carrier or the like that itself does not have activity.

Hereinafter, the present invention will be described in detail.

The present invention provides a composition for inducing stem cells to differentiate into astrocytes, comprising CPI-455 as an active ingredient.

In the present invention, “CPI-455” is named 4,7-dihydro-6-(1-methylethyl)-7-oxo-5-phenyl-pyrazolo[1,5-a]pyrimidine-3-carbonitrile (Cas No. 1628208-23-0) and is represented by Chemical Formula 1 below. Additionally, CPI-455 may be obtained as a commercial product or prepared by a known method (Benjamin R. Leadem. NOVEL HISTONE DEMETHYLASE INHIBITORS SYNERGISTICALLY).

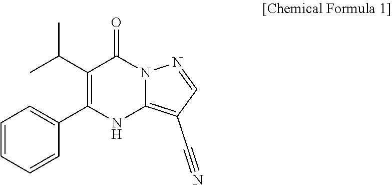 [SMILES: CC(C)c1c(-c2ccccc2)[nH]c2c(C#N)cnn2c1=O]

In the present invention, the CPI-455 may be included at a concentration of 0.1 to 100 μM, 1 to 90 μM, 1 to 80 μM, 1 to 70 μM, 1 to 60 μM, 1 to 55 μM, 1 to 50 μM, 1 to 45 μM, 1 to 40 μM, 10 to 90 μM, 10 to 80 μM, 10 to 70 μM, 10 to 60 μM, 10 to 50 μM, 20 to 80 μM, 30 to 70 μM, 40 to 60 μM, 43 to 60 μM, 45 to 60 μM, 47 to 60 μM, 50 to 58 μM, 50 to 55 μM, 50 to 54 μM, 50 to 52μ, 46μ, 47μ, 48μ, 49μ, 50μ, 51μ, 52μ, 53μ, or 54 μM based on the total composition, but is not limited thereto.

According to one example of the present invention, the CPI-455 induces differentiation of neural stem cells into astrocytes by increasing BMP2 secretion and promoting STAT3 phosphorylation, and is characterized by satisfying one or more of the following characteristics:
(a) promoting phosphorylation of signal transducer and activator of transcription 3 (STAT3); and
(b) increasing secretion of bone morphogenic protein 2 (BMP2).

In the present invention, “BMP2 (Bone morphogenic protein 2)” is one of the TGF-beta family cytokines, a polypeptide dimer composed of 114 amino acids, known to interact with fibroblast growth factors to induce not only the formation of chondrocyte and osteoblast precursors but also the development of epidermal tissue, and further to induce differentiation of neural cells.

In the present invention, “STAT3 (Signal transducer and activator of transcription 3)” is one member of the STAT family, which exists in an inactive form in the cytoplasm and, upon stimulation by various cytokines, growth factors, or hormones, becomes phosphorylated, forms a dimer, and then translocates into the nucleus to deliver signals as a transcription factor. STAT3 regulates the expression of various genes, thereby inducing cell differentiation, survival, proliferation, and apoptosis, and is particularly known to be upregulated in many tumor cells. The activity of STAT3 is regulated by phosphorylation of Janus kinase 2 (JAK2), which is an upstream kinase that promotes STAT3 phosphorylation, by the expression level of suppressor of cytokine signaling 3 (SOCS3) protein that binds to JAK2 to inhibit its activity, and by the activity of the tyrosine phosphatase SH2 domain-containing protein tyrosine phosphatase (SHP-1).

In the present invention, the term “stem cell” is not particularly limited as long as it is in an undifferentiated state and has the ability to proliferate indefinitely and to differentiate into astrocytes. Specifically, stem cells may include embryonic stem cells, adult stem cells, embryonic germ stem cells, and embryonic tumor stem cells, but are not limited thereto. According to one example of the present invention, the stem cells may be neural stem cells.

In the present invention, the term “astrocyte” may refer to a cell in which the expression level of GFAP, an astrocyte marker, is increased, but is not limited thereto.

Additionally, the present invention provides a method for differentiating stem cells into astrocytes, the method comprising treating the stem cells with the composition for inducing stem cells to differentiate into astrocytes according to the present invention and culturing the stem cells.

According to one example of the present invention, the astrocytes prepared by the method of differentiating stem cells into astrocytes according to the present invention may express GFAP (Glial fibrillary acidic protein), an astrocyte marker, and may have an increased GFAP expression level compared to stem cells.

In the present invention, “culturing” may be carried out in any one of cell culture media selected from DMEM (Dulbecco's Modified Eagle Medium), DMEM/F12 (Dulbecco's Modified Eagle Medium: Nutrient Mixture F-12), RPMI1640 (Roswell Park Memorial Institute 1640), MEM (Minimum Essential Medium), and Ham F10, and sodium pyruvate, glutamine, and the like may be additionally added to the culture medium. The culture period may be 1 to 10 days, 1 to 8 days, 1 to 6 days, 2 to 6 days, 3 to 6 days, 3 to 5 days, or 3 to 4 days, but is not limited thereto.

The “cell culture medium” or “culture medium” refers to a mixture for differentiation and proliferation of cells such as astrocytes in vitro, which contains essential elements for growth and proliferation of cells such as sugars, amino acids, various nutrients, serum, growth factors, and minerals, and particularly, in the present invention, the “cell culture medium” is a medium for differentiation and proliferation of astrocytes. In the present invention, the culturing means differentiation and proliferation of astrocytes.

In the present invention, the term “medium” may include any basic medium known in the art without limitation. The basic medium may be artificially synthesized and prepared, and commercially available media may include, for example, DMEM (Dulbecco's Modified Eagle's Medium), MEM (Minimal Essential Medium), BME (Basal Medium Eagle), RPMI 1640, F-10, F-12, α-MEM (α-Minimal Essential Medium), G-MEM (Glasgow's Minimal Essential Medium), and Iscove's Modified Dulbecco's Medium, but are not limited thereto.

Additionally, the culture medium of the present invention may further include a nutrient mixture. The nutrient mixture is a mixture including various amino acids, vitamins, and inorganic salts generally used in cell culture, and may be prepared by mixing the amino acids, vitamins, and inorganic salts, or a commercially available nutrient mixture may be used. Commercially available nutrient mixtures may include, for example, B27, N2, F-12, M199, MCDB110, MCDB202, and MCDB302, but are not limited thereto.

Additionally, the culture medium of the present invention may not include growth factors. The growth factors may be, for example, EGF or FGF2, but are not limited thereto.

In the present invention, the culture solution for culturing stem cells includes all media culture solutions commonly used for astrocyte differentiation in the art. The culture solution used for culturing generally includes carbon sources, nitrogen sources, and trace element components.

More specifically, the method for differentiating stem cells into astrocytes may include a step of culturing the stem cells in a differentiation medium treated with a composition comprising CPI-455 as an active ingredient to differentiate the stem cells into astrocytes.

The stem cells may include embryonic stem cells, adult stem cells, embryonic germ stem cells, and embryonic tumor stem cells, but are not limited thereto.

The differentiation medium may include one or more selected from the group consisting of an antibiotic-antimycotic and B27, but is not limited thereto.

The differentiation medium may include one or more selected from the group consisting of 0.1 to 5% (v/v) of an antibiotic-antimycotic and 0.5 to 5% (v/v) of B27, but is not limited thereto. The differentiation medium may not include growth factors EGF and FGF2, but is not limited thereto.

Additionally, the present invention provides a kit for inducing stem cells to differentiate into astrocytes, comprising CPI-455 or the composition for inducing stem cells to differentiate into astrocytes according to the present invention.

The kit is not limited to the above-described components and may include other reagents or instruments. For example, it may include a culture plate for culturing target cells or a reagent for evaluating the differentiation state into astrocytes, and may further comprise the stem cells to be cultured as the target.

The kit according to the present invention may be provided in the form of a single container containing, in an appropriate amount and/or form, an additive for differentiation induction medium, CPI-455, a nutrient mixture, a growth factor, a basal medium, and other reagents, or may be provided in separate containers.

The kit according to the present invention may further comprise an instruction manual describing the procedures for carrying out the above-described method according to the present invention.

Additionally, the present invention provides a use of CPI-455 or the composition for inducing stem cells to differentiate into astrocytes according to the present invention for inducing stem cells to differentiate into astrocytes.

Additionally, the present invention provides a pharmaceutical composition for preventing or treating a neurodegenerative disorder, comprising CPI-455 or a pharmaceutically acceptable salt thereof as an active ingredient.

The CPI-455 according to the present invention may be used as it is or in the form of a pharmaceutically acceptable salt thereof.

In the present invention, the term “pharmaceutically acceptable salt” includes a salt derived from a pharmaceutically acceptable inorganic acid, organic acid, or base.

Examples of suitable acids include hydrochloric acid, hydrobromic acid, sulfuric acid, nitric acid, perchloric acid, propionic acid, maleic acid, tartaric acid, gluconic acid, trifluoroacetic acid, fumaric acid, maleic acid, phosphoric acid, glycolic acid, lactic acid, salicylic acid, succinic acid, p-toluenesulfonic acid, tartaric acid, acetic acid, citric acid, methanesulfonic acid, formic acid, benzoic acid, malonic acid, naphthalene-2-sulfonic acid, benzenesulfonic acid, hydrobromic acid, hydroiodic acid, nitrous acid, benzoic acid, citric acid, and lactic acid. An acid addition salt may be prepared by conventional methods, for example, by dissolving the compound in an excess of an aqueous solution of the acid and precipitating the salt using a water-miscible organic solvent such as methanol, ethanol, acetone, or acetonitrile. In addition, the salt may be prepared by heating an equimolar amount of the compound and an acid or alcohol in water, followed by evaporating the mixture to dryness, or by suction filtering the precipitated salt.

A salt derived from a suitable base may include alkali metals such as sodium and potassium, alkaline earth metals such as magnesium, and ammonium, but is not limited thereto. An alkali metal or alkaline earth metal salt may be obtained, for example, by dissolving the compound in an excess of an alkali metal hydroxide or alkaline earth metal hydroxide solution, filtering the insoluble compound salt, and evaporating and drying the filtrate. In this case, metal salts, particularly sodium, potassium, or calcium salts, are pharmaceutically preferred, and the corresponding silver salts may be obtained by reacting the alkali metal or alkaline earth metal salts with a suitable silver salt (e.g., silver nitrate).

The term “neurodegenerative disorder” used in the present invention is not particularly limited in type. Non-limiting examples of the neurodegenerative disorder include Alzheimer's disease, amyotrophic lateral sclerosis, adrenoleukodystrophy, Alexander's disease, Alper's disease, ataxia telangiectasia, Batten's disease, bovine spongiform encephalopathy (BSE), Canavan's disease, corticobasal degeneration, Creutzfeldt-Jakob disease, dementia with Lewy bodies, fatal familial insomnia, frontotemporal lobar degeneration, Huntington's disease, Kennedy's disease, Krabbe's disease, Lyme disease, Machado-Joseph disease, multiple sclerosis, multiple system atrophy, neuroacathocytosis, Niemann-Pick's disease, Parkinson's disease, Pick's disease, primary lateral sclerosis, progressive supranuclear palsy, Refsum's disease, Sandhoff's disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome, but are not limited thereto.

The content of the compound in the composition of the present invention may be appropriately adjusted depending on the symptoms of the disease, the degree of progression of the symptoms, the condition of the patient, and the like, and may be, for example, 0.0001 to 99.9% by weight, 0.001 to 80% by weight, or 0.01 to 50% by weight based on the total weight of the composition, but is not limited thereto. The content ratio is a value based on the dry weight obtained after removal of the solvent.

In the present invention, the term “pharmaceutical composition” refers to a composition prepared for the purpose of preventing or treating a disease, and may be formulated and used in various forms according to conventional methods. For example, it may be formulated into oral dosage forms such as powders, granules, tablets, capsules, suspensions, emulsions, or syrups, and may be formulated and used in the form of external preparations, suppositories, or sterile injectable solutions.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

Additionally, the present invention provides a method for preventing or treating a neurodegenerative disorder, the method comprising administering CPI-455, or a composition comprising CPI-455 as an active ingredient, to a subject in need thereof.

Additionally, the present invention provides a use of CPI-455, or a composition comprising CPI-455 as an active ingredient, for preventing or treating a neurodegenerative disorder.

Additionally, the present invention provides a use of CPI-455, or a composition comprising CPI-455 as an active ingredient, for preparing a therapeutic agent for neurodegenerative disorders.

As used herein, the “subject” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

Furthermore, the pharmaceutical composition of the present invention may be administered in combination with a known compound having a preventive or therapeutic effect on a neurodegenerative disorder.

In the present invention, stem cells were induced to differentiate into astrocytes using CPI-455. The differentiated astrocytes have a very high potential for use as a cell therapy agent for patients.

Therefore, the present invention provides a cell therapy agent for preventing or treating a neurodegenerative disorder, comprising astrocytes prepared by the method of differentiating stem cells into astrocytes according to the present invention.

Additionally, the present invention provides a method for preventing or treating a neurodegenerative disorder, the method comprising administering the cell therapy agent according to the present invention to a subject in need thereof.

Additionally, the present invention provides a use of the cell therapy agent according to the present invention for preventing or treating a neurodegenerative disorder.

The term “cell therapy agent” of the present invention refers to a pharmaceutical (according to U.S. FDA regulations) comprising cells and tissues prepared through isolation, culture, and special manipulation from humans, which is used for the purposes of treatment, diagnosis, and prevention, and means a pharmaceutical in which such cells are used for the treatment, diagnosis, and prevention of diseases through a series of processes such as proliferating and selecting living autologous, allogenic, or xenogenic cells in vitro, or changing the biological characteristics of cells by other methods, in order to restore the function of cells or tissues.

The administration route of the cell therapy agent according to the present invention may be any general route as long as it can reach the target tissue. Parenteral administration, for example, intraperitoneal administration, intravenous administration, intramuscular administration, subcutaneous administration, or intradermal administration, may be employed, but is not limited thereto.

The cell therapy agent may be formulated in an appropriate form together with a pharmaceutically acceptable carrier generally used in cell therapy. The term “pharmaceutically acceptable” refers to a composition that is physiologically acceptable and does not generally cause allergic reactions such as gastrointestinal disorders, dizziness, or similar reactions when administered to humans. Examples of pharmaceutically acceptable carriers include, for example, parenteral carriers such as water, suitable oils, saline, aqueous glucose, and glycols, and may further include stabilizers and preservatives. Suitable stabilizers include antioxidants such as sodium bisulfite, sodium sulfite, or ascorbic acid. Suitable preservatives include benzalkonium chloride, methyl- or propyl-paraben, and chlorobutanol. Other pharmaceutically acceptable carriers may be referenced from the literature (Remington's Pharmaceutical Sciences, 19th ed., Mack Publishing Company, Easton, PA, 1995).

Additionally, the composition may be administered by any device that allows the cell therapy agent to move to the target cells.

The cell therapy agent according to the present invention may include a therapeutically effective amount of astrocytes for the treatment of a disease. The term “therapeutically effective amount” means the amount of an active ingredient or therapeutic agent that induces a biological or medical response in a tissue system, animal, or human as considered appropriate by a researcher, veterinarian, physician, or other clinician, and includes an amount that induces alleviation of the symptoms of the disease or disorder being treated.

It will be apparent to those skilled in the art that the cells included in the cell therapy agent according to the present invention will vary depending on the desired effect. Therefore, the optimal content of the cell therapy agent may be easily determined by those skilled in the art and may be adjusted depending on various factors including the type of disease, severity of the disease, content of other components contained in the composition, type of formulation, and the age, weight, general health status, sex, and diet of the patient, administration time, administration route, excretion rate of the therapeutic agent, treatment period, and concomitant medications. It is important to include an amount that provides the maximum effect with the minimum amount without side effects, considering all of the above factors. For example, the daily dose of astrocytes of the present invention may be 1.0×10⁴ to 1.0×10¹¹ cells/kg body weight, preferably 1.0×10⁵ to 1.0×10⁹ cells/kg body weight, administered once or divided into several administrations. However, it should be understood that the actual dose of the active ingredient should be determined in light of various relevant factors such as the disease to be treated, severity of the disease, administration route, and the patient's body weight, age, and sex, and therefore, the above dose is not intended to limit the scope of the present invention in any way.

The present invention provides a therapeutic method comprising administering a therapeutically effective amount of the cell therapy agent according to the present invention to a mammal. The term “mammal” as used herein refers to a mammal that is the subject of treatment, observation, or experiment, and preferably refers to a human.

Additionally, the present invention provides a food composition for preventing or improving a neurodegenerative disorder, comprising CPI-455 or a food-acceptable salt thereof as an active ingredient, wherein the food composition may be a health functional food composition.

In the present invention, the term “food-acceptable salt” includes a salt derived from a food-acceptable organic acid, inorganic acid, or base. When the CPI-455 of the present invention is used as a food additive, it may be added as it is or used together with other foods or food ingredients, and may be appropriately used according to conventional methods. The amount of active ingredient mixed may be suitably determined according to the purpose of use (prevention, health or therapeutic treatment).). In general, when a food or beverage is prepared, the CPI-455 of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

In the present specification, the term “health functional food” is the same term as food for special health use (FoSHU), and refers to a food processed to efficiently exhibit bioregulatory functions in addition to nutritional supply and having high medical or therapeutic effects, and the food may be manufactured in various forms such as tablets, capsules, powders, granules, liquids, or pills in order to obtain useful effects for preventing or improving neurodegenerative disorders.

The health functional food of the present invention may be prepared by methods conventionally used in the art, and in the preparation thereof, raw materials and ingredients conventionally added in the art may be used. In addition, unlike general medicines, since it is prepared from food materials, it has the advantage of not causing side effects that may occur with long-term administration of medicines, and may have excellent portability.

### MODES OF THE INVENTION

Hereinafter, preferred examples are presented to help understand the present invention. However, the following examples are provided only to help understand the present invention more easily, and the contents of the present invention are not limited by the following examples.

### Experimental Methods

### 1. Cell Culture, Chemical Treatment, and Sampling

All animal experiments were approved by the Institutional Animal Care and Use Committee of Chung-Ang University and were conducted in accordance with the guidelines for the care and use of laboratory animals of the National Institutes of Health.

Neural stem cells (NSCs) were cultured and harvested as previously reported (Faseb J 2018, 32 (2), 1108-1119). Specifically, the cells were harvested by dissecting the cerebral cortex from embryos of 14-day pregnant Sprague-Dawley rats (Orient Bio, Seongnam, South Korea). The cells were incubated for 6 days at 37° C. in 5% CO₂ in Dulbecco's modified Eagle's medium (DMEM/F12) (Cat no. 12500-062, Gibco, Grand Island, NY, U.S.A.) containing Ham's nutrient mixture F-12 supplemented with 1% antibiotic-antimycotic (Cat no. 15240-062, Gibco), 2% B-27 supplement (Cat no. 17504-044, Gibco), 20 ng/ml EGF (Cat no. GF144, Chemicon, Burlington, MA, U.S.A.), and 20 ng/ml FGF2 (Cat no. GF003AF, Chemicon), so as to form neurospheres. The neurospheres were then dissociated into single cells using Accutase (Cat No. SCR005, Sigma-Aldrich, St. Louis, MO, U.S.A.). The cells were then attached to culture plates for one day in medium enriched with EGF and FGF2. Next, the NSCs were treated with DMEM/F12 medium containing 1% antibiotic-antimycotic, 2% B-27 supplement, and either DMSO (Cat no. 472301, Sigma-Aldrich) or various concentrations of CPI-455 (Cat no. HY-100421, Medchemexpress, Princeton, NJ, U.S.A.) dissolved in DMSO. The medium was replaced every 2 days. Cells for western blot and immunostaining were cultured for 4 days after treatment with CPI-455. Cells for RT-qPCR and ChIP-qPCR were cultured for 3 days after treatment with CPI-455.

### 2. Western Blot

The cells were washed with PBS and lysed on ice using NP-40 lysis buffer. The lysates were centrifuged at 16,000 g for 30 minutes to purify the total protein. Proteins were separated by electrophoresis on an 8-10% polyacrylamide gel and transferred onto an Immobilon-P PVDF membrane (Cat no. PVH00010, Millipore, Burlington, MA, U.S.A.). The membrane was blocked with 5% non-fat dry milk in TBS/0.03% Tween 20 or with 5% bovine serum albumin in TBS/0.1% Tween 20.

The membrane was incubated overnight with the following primary antibodies: TUBB3 (1:3000; Cat no. T5076, Sigma-Aldrich), GFAP (1:9000; Cat no. G9269, Sigma-Aldrich), OLIG2 (1:2000; Cat no. AB9610, Millipore), GAPDH (1:1000; Cat no. sc-32233, Santa Cruz Biotechnology, Dallas, TX, U.S.A.), STAT3 (1:2000; Cat no. 4904, Cell Signaling Technology, Danvers, MA, U.S.A.), and pSTAT3 (1:2000; Cat no. 9145, Cell Signaling Technology).

The following secondary antibodies were applied for 1 hour: Anti-mouse IgG F(ab′) 2 (1:5000; Cat no. ADI-SAB-100, Enzo, Farmingdale, NY, U.S.A.) and Anti-Rabbit IgG (1:5000; Cat no. ADI-SAB-300-J, Enzo).

Chemiluminescence was detected on CP-BU new film (Agfa, Mortsel, Belgium) using western blotting luminol reagent (Cat no. sc-2048, Santa Cruz Biotechnology). Band intensity was quantified using ImageJ version 2.3.0. GAPDH (Glyceraldehyde-3-phosphate dehydrogenase) was measured as a housekeeping protein.

### 3. Immunocytochemistry (ICC)

Immunocytochemistry was performed as previously reported (Faseb J 2018, 32 (2), 1108-1119). Specifically, the cells were fixed in 4% paraformaldehyde solution (Cat no. P231, Biosesang, Seongnam, South Korea) for 30 minutes. The cells were blocked with 5% goat serum (Cat no. S26, Millipore) diluted in 0.2% Triton X-100 (Cat no. 0694-1L, VWR International, Radnor, PA, U.S.A.).

The cells were stained for 1 hour with the following primary antibodies: TUBB3 (1:1000; Cat no. T5076, Sigma-Aldrich) and GFAP (1:500; Cat no. Z0334, Dako, Santa Clara, CA, U.S.A.). Next, the following secondary antibodies were applied for 30 minutes: Alexa488 goat anti-mouse IgG (1:1000; Cat no. A21131, Invitrogen, Waltham, MA, U.S.A.) and Cy3 goat anti-rabbit IgG (1:1000; Cat no. 111-165-144, Jackson, West Grove, PA, U.S.A.).

The nuclei were stained by treating the cells with 0.01% DAPI (4′,6-diamidino-2-phenylindole) (Cat No. D9542, Sigma-Aldrich) in PBS solution for 5 minutes. Fluorescent images were captured using a Leica DM IL inverse fluorescent microscope and LAS X software (Leica, Wetzlar, Germany).

### 4. Reverse Transcriptase-Quantitative Polymerase Chain Reaction (RT-qPCR)

RNA was extracted using TRIzol® reagent (Cat no. 15596018, Thermo Fisher, Waltham, MA, U.S.A.) and dissolved in diethylpyrocarbonate-treated water (Cat no. C-9030, Bioneer, Daejeon, South Korea). Complementary DNA (cDNA) was synthesized from 1 μg of RNA per sample using a QuantiTect reverse transcriptase kit (Cat no. 205311, Qiagen, Hilden, Germany). The cDNA was mixed with iQ™SYBR® Green supermix (Cat no. 1708880, Bio-Rad, Hercules, CA, U.S.A.) and each forward and reverse primer (Cosmo Genetech, Seoul, South Korea). The primers used are shown in Table 1. qPCR was performed for 40 cycles under the following conditions using a CFX Connect thermal cycler (Bio-Rad): 95° C. for 10 seconds, 58° C. for 15 seconds, and 72° C. for 20 seconds.

TABLE 1

| | | | |
|---|---|---|---|
| RT-qPCR | gapdh | Forward | 5′-AGTTCAACGGCACAGTCAAG-3′ |
| | | (SEQ ID NO: 1) | |
| | | Reverse | 5′-GTGGTGAAGACGCCAGTAGA-3 |
| | | (SEQ ID NO: 2) | |
| | tubb3 | Forward | 5′-AGCCCTCTACGACATCTGCT-3′ |
| | | (SEQ ID NO: 3) | |
| | | Reverse | 5′-ATTGAGCTGACCAGGGAATC-3′ |
| | | (SEQ ID NO: 4) | |
| | gfap | Forward | 5′-AGCGGCTCTGAGAGAGATTC-3′ |
| | | (SEQ ID NO: 5) | |
| | | Reverse | 5′-AGCAACGTCTGTGAGGTCTG-3′ |
| | | (SEQ ID NO: 6) | |
| | il-6 | Forward | 5′-TGTTCTCAGGGAGATCTTGG-3′ |
| | | (SEQ ID NO: 7) | |
| | | Reverse | 5′-TTTGGAAGCATCCATCATTT-3′ |
| | | (SEQ ID NO: 8) | |
| | lif | Forward | 5′-AAGTTGGTCGAGCTGTATCG-3′ |
| | | (SEQ ID NO: 9) | |
| | | Reverse | 5′-GAGTTTGATCTGGAGGCTCA-3′ |
| | | (SEQ ID NO: 10) | |
| | cntf | Forward | 5′-GCAAGGAAGATTCGTTCAGA-3′ |
| | | (SEQ ID NO: 11) | |
| | | Reverse | 5′-TCATCTCACTCCAACGATCA-3′ |
| | | (SEQ ID NO: 12) | |
| | bmp2 | Forward | 5′-AACAAATGCAGGAAGCTTTG-3′ |
| | | (SEQ ID NO: 13) | |
| | | Reverse | 5′-CCTGGTGTCCAATAGTCTGG-3′ |
| | | (SEQ ID NO: 14) | |

### 5. Chromatin Immunoprecipitation (ChIP)

Chromatin immunoprecipitation was performed as described in Faseb J 2018, 32 (2), 1108-1119 with modifications of the method described in Nature 2007, 450 (7168), 415-419. Specifically, the cells were fixed with 1% formaldehyde (Cat no. BP531, Fisher Scientific, Waltham, MA, U.S.A.) for 10 minutes and collected by scraping. The cell suspension was centrifuged at 700 g for 5 minutes and lysed in buffer. Chromatin was sheared to 0.5-1 kb fragments using a VCX-500 sonicator (Sonics & Materials, Newtown, CT, U.S.A.) equipped with a 2 mm stepped microtip. One percent of the volume of each sample was separated as 1% input. Chromatin was immunoprecipitated using a rabbit H3K4me3 antibody (Cat no. 07-473, Millipore) and protein A-agarose beads (Cat No. 16-157, Millipore). The chromatin was reverse-crosslinked by overnight incubation at 65° C. in 200 mM NaCl. DNA fragments were purified using a QIAquick PCR Purification Kit (Cat no. 28104, Qiagen). qPCR was performed as described in section 4. RT-qPCR. The 1% input of each sample was reverse-crosslinked, purified, and subjected to qPCR in parallel with the immunoprecipitated samples.

### 6. Statistical Analysis

Results in bar charts were expressed as mean±SEM or SD. To evaluate the statistical significance of results between two groups, a two-tailed Student's t-test was performed, with the threshold set at p<0.05 vs. control. To evaluate the statistical significance of results among three or more groups, one-way ANOVA was performed, followed by Dunnett's multiple comparisons test using GraphPad Prism 7.00.

### EXAMPLES

### Example 1. CPI-455 Inducing Astrocytic Differentiation in NCS

NSCs proliferate in the presence of an epidermal growth factor (EGF) and fibrous growth factor-2 (FGF2), and in the absence of growth factors in a medium, NSCs differentiate into neurons and astrocytes. To examine the effect of CPI-455 on NSC differentiation, brain cortical tissue was collected from embryonic day 14 (E14) Sprague-Dawley rats and NSCs were proliferated in the presence of EGF and FGF2 for one week. Then, growth factor-free NSCs were treated with CPI-455 or DMSO (0.1%) control to induce differentiation for 4 days.

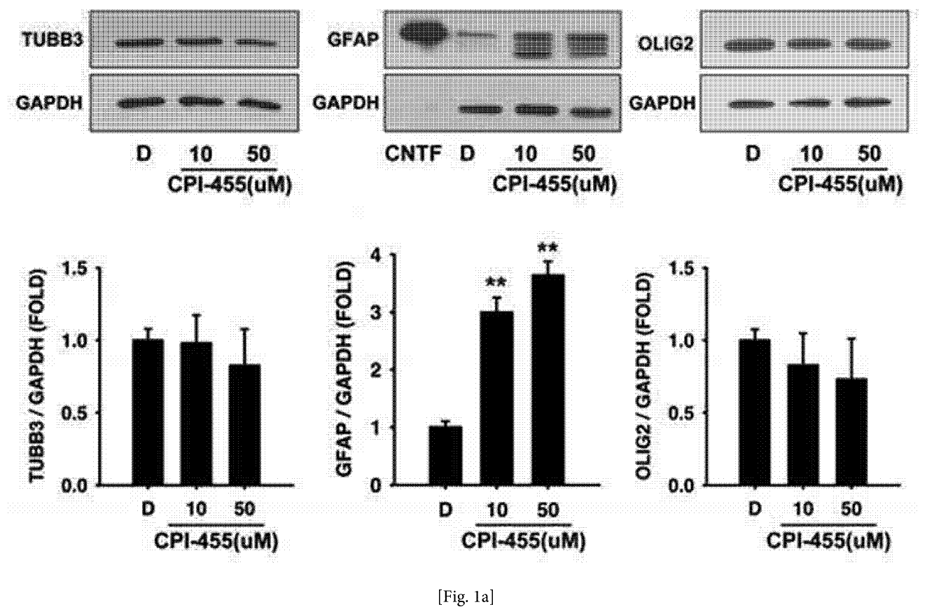

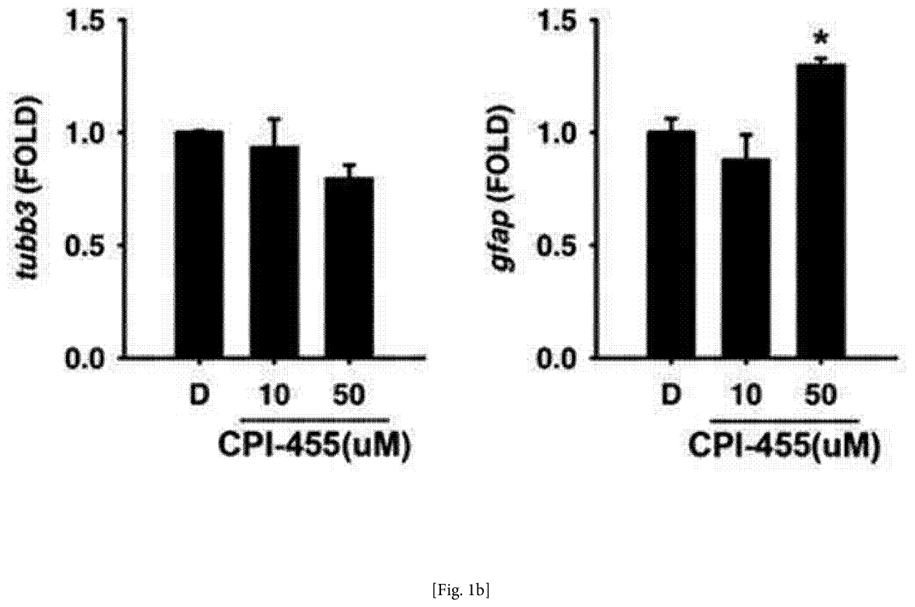

As a result of performing a western blot targeting a neuronal marker protein TUBB3, an astrocyte marker protein GFAP, and an oligodendrocyte marker protein OLIG2 in differentiating neurons, as shown in FIG. 1A, it could be confirmed that CPI-455 significantly increased GFAP protein expression compared to the control, but there was no significant change in TUBB3 or OLIG2 expression. RT-qPCR results showed that gfap mRNA expression was significantly increased in cells treated with 50 μM CPI-455 compared to the control, as shown in FIG. 1B. This means that treatment with CPI-455 during NSC differentiation increases astrocytogenesis.

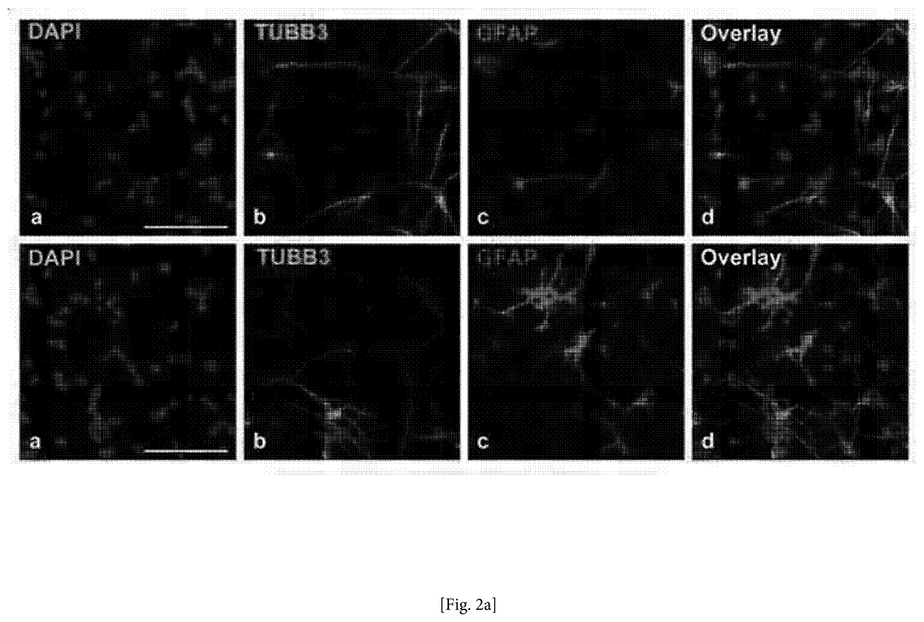

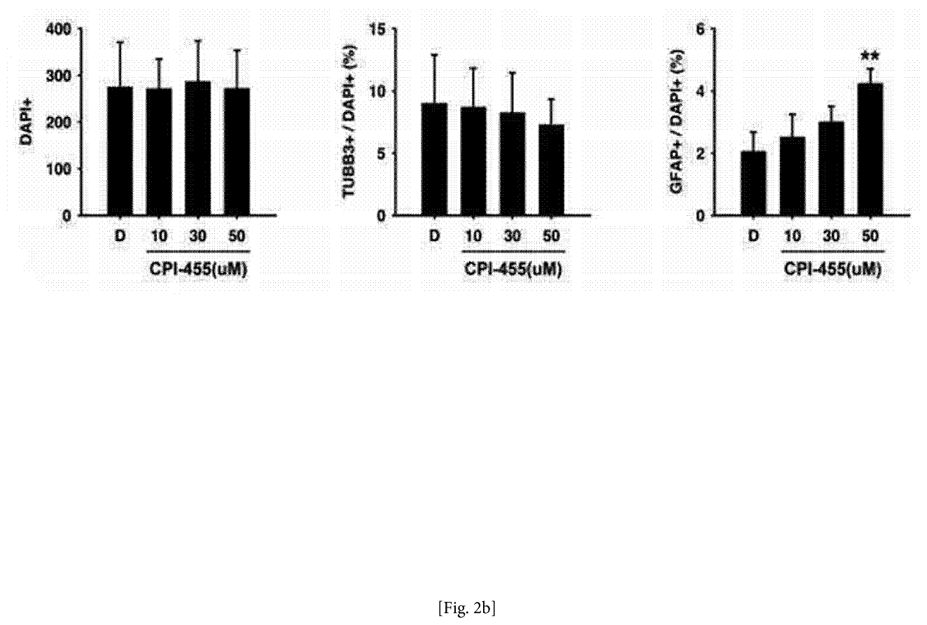

In addition, immunocytochemistry (ICC) was performed to assess astrocyte percentages during NSC differentiation to determine whether the induction of GFAP was due to an increase in astrocytes or an increase in protein expression in individual cells. As a result, as shown in FIGS. 2A and 2B, it could be observed that when differentiating NSCs were treated with 50 μM CPI-455, the number of GFAP-positive cells significantly increased. In contrast, the percentage of TUBB3-positive cells did not show any significant difference between the control and the CPI-455-treated groups. No significant difference in the number of DAPI-positive cells was observed between the control and the CPI-455-treated groups. This means that CPI-455 treatment during NSC differentiation enhances astrocytogenesis while not inducing cytotoxicity.

### Example 2. CPI-455 Inducing Astrocytogenesis Through BMP2-STAT3 Signaling

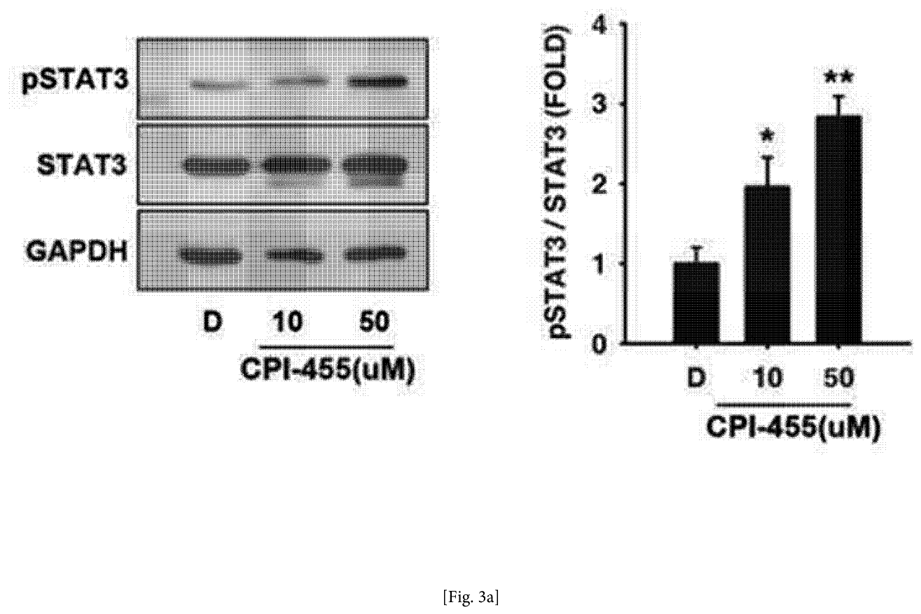

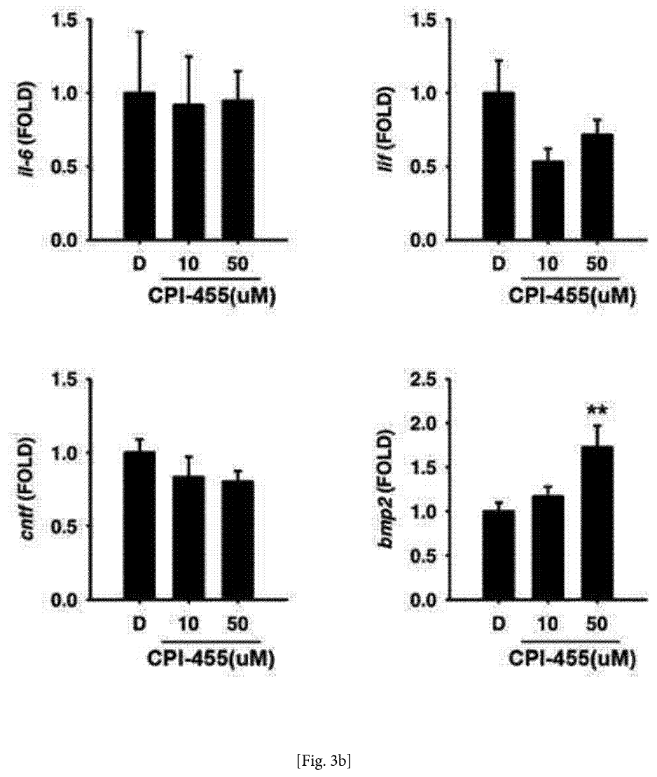

To confirm the mechanism by which astrocytes are generated in NSCs treated with CPI-455, Western blot was performed to determine STAT3 expression and phosphorylation. As a result, as shown in FIG. 3A, it could be confirmed that treatment with 50 μM CPI-455 significantly increased phosphorylated STAT3 (pSTAT3) with respect to total STAT3 in differentiating neurons. The Janus kinase (JAK)/STAT pathway is one of the signaling pathways involved in various cellular events including cellular differentiation, inflammation and regeneration. It was previously reported that the JAK/STAT pathway is involved in regulating astrocytic differentiation in NSCs. It was shown that STAT3 was activated during the generation of ciliary neurotrophic factor (CNTF)-induced astrocytes, and disruption of STAT3 using dominant negative mutations suppressed astrocytogenesis. STAT3 has also been reported to be a regulator of proliferation, differentiation and self-renewal in various stem cells and cancer cells. The induction of STAT3 phosphorylation indicates that the JAK/STAT3 signaling pathway is activated during CPI-455-induced astrocytogenesis.

The cell fate and differentiation of NSCs are regulated by various cytokines. The JAK/STAT pathway may be activated by cytokines of the interleukin-6 (IL-6) family, including leukemia inhibitory factor (LIF) and CNTF. BMP2, a transforming growth factor-beta (TGF-β) family cytokine, may also induce astrocytic differentiation in fetal mouse brain cells and NSCs. Therefore, to investigate cytokine expression associated with astrocytic differentiation and STAT3 signaling in CPI-455-treated neurons, the levels of il-6, lif, cntf, and bmp2 were confirmed by RT-qPCR. As a result, as shown in FIG. 3B, it was confirmed that bmp2 significantly increased in neurons treated with 50 μM CPI-455.

Interestingly, among the investigated cytokines known to induce astrocytogenesis in NSCs, bmp2 was identified as the only cytokine mRNA that showed a significant increase. Since BMP2 was reported to induce developmental changes toward astrocytogenesis through the activation of the Smad pathway, CPI-455-induced astrocytogenesis may be due to the activation of the BMP2 signaling pathway. In general, LIF and BMP2 synergistically induce astrocytogenesis through the activation of the JAK-STAT3 and Smad pathways, respectively. However, increased STAT3 phosphorylation and astrocytogenesis in CPI-455-induced astrocytogenesis were observed with an increase in bmp2 expression without an increase in lif expression. It was speculated that such a discrepancy between lif expression and STAT3 phosphorylation might be due to a non-canonical BMP2-STAT3 signaling pathway involving phosphorylation of STAT3. Indeed, the BMP2-STAT3 pathway has been reported to be a pathway capable of inducing astrocytogenesis without LIF stimulation. The treatment of human oral carcinoma cells with recombinant BMP2 also increased STAT3 phosphorylation, supporting the assertion that BMP2 stimulation increases STAT3 phosphorylation during signal transduction.

In consideration of these results on STAT3 phosphorylation and BMP2 signaling, it was speculated that the astrocytogenic effect of CPI-455 was induced by the activation of a Gfap promoter through the non-canonical BMP2-STAT3 signaling pathway.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

By exhibiting a technique for inducing stem cells to differentiate into astrocytes using CPI-455, the present invention is expected not only to be able to induce differentiation into astrocytes without the use of existing stem cell stem cell differentiation promoters, but also to be able to effectively treat neurodegenerative disorders using differentiated astocytes, and thus has industrial applicability.

1. A method for differentiating stem cells into astrocytes, the method comprising treating the stem cells with CPI-455, or a composition comprising CPI-455 as an active ingredient, and culturing the stem cells.

2. The method of claim 1, wherein the stem cells are one or more selected from the group consisting of embryonic stem cells, adult stem cells, embryonic germ stem cells, and embryonic tumor stem cells.

3. The method of claim 1, wherein the CPI-455 is included at a concentration of 0.1 μM to 100 μM based on the total composition comprising the CPI-455 as an active ingredient.

4. The method of claim 1, wherein the CPI-455 satisfies one or more of the following characteristics:
(a) promoting phosphorylation of signal transducer and activator of transcription 3 (STAT3); and
(b) increasing secretion of bone morphogenic protein 2 (BMP2).

5. (canceled)

6. The method of claim 1, wherein the astrocytes express glial fibrillary acidic protein (GFAP).

7. (canceled)

9. A method for alleviating or treating a neurodegenerative disorder, the method comprising administering a pharmaceutically effective amount of CPI-455, or a composition comprising CPI-455 as an active ingredient, to a subject in need thereof.

10. The method of claim 9, wherein the neurodegenerative disorder is one or more selected from the group consisting of Alzheimer's disease, amyotrophic lateral sclerosis, adrenoleukodystrophy, Alexander's disease, Alper's disease, ataxia telangiectasia, Batten's disease, bovine spongiform encephalopathy (BSE), Canavan's disease, corticobasal degeneration, Creutzfeldt-Jakob disease, dementia with Lewy bodies, fatal familial insomnia, frontotemporal lobar degeneration, Huntington's disease, Kennedy's disease, Krabbe's disease, Lyme disease, Machado-Joseph disease, multiple sclerosis, multiple system atrophy, neuroacathocytosis, Niemann-Pick's disease, Parkinson's disease, Pick's disease, primary lateral sclerosis, progressive supranuclear palsy, Refsum's disease, Sandhoff's disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

11.-13. (canceled)

14. A method for alleviating or treating a neurodegenerative disorder, the method comprising administering a pharmaceutically effective amount of a cell therapy agent comprising astrocytes prepared by the method of claim 1, to a subject in need thereof.

15. The method of claim 14, wherein the neurodegenerative disorder is one or more selected from the group consisting of Alzheimer's disease, amyotrophic lateral sclerosis, adrenoleukodystrophy, Alexander's disease, Alper's disease, ataxia telangiectasia, Batten's disease, bovine spongiform encephalopathy (BSE), Canavan's disease, corticobasal degeneration, Creutzfeldt-Jakob disease, dementia with Lewy bodies, fatal familial insomnia, frontotemporal lobar degeneration, Huntington's disease, Kennedy's disease, Krabbe's disease, Lyme disease, Machado-Joseph disease, multiple sclerosis, multiple system atrophy, neuroacathocytosis, Niemann-Pick's disease, Parkinson's disease, Pick's disease, primary lateral sclerosis, progressive supranuclear palsy, Refsum's disease, Sandhoff's disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

16.-18. (canceled)


SEQUENCE LISTING (14 sequences)

SEQ ID NO: 1 - DNA, 20 bp, synthetic construct
  source 1..20: mol_type = other DNA
agttcaacggcacagtcaag

SEQ ID NO: 2 - DNA, 20 bp, synthetic construct
  source 1..20: mol_type = other DNA
gtggtgaagacgccagtaga

SEQ ID NO: 3 - DNA, 20 bp, synthetic construct
  source 1..20: mol_type = other DNA
agccctctacgacatctgct

SEQ ID NO: 4 - DNA, 20 bp, synthetic construct
  source 1..20: mol_type = other DNA
attgagctgaccagggaatc

SEQ ID NO: 5 - DNA, 20 bp, synthetic construct
  source 1..20: mol_type = other DNA
agcggctctgagagagattc

SEQ ID NO: 6 - DNA, 20 bp, synthetic construct
  source 1..20: mol_type = other DNA
agcaacgtctgtgaggtctg

SEQ ID NO: 7 - DNA, 20 bp, synthetic construct
  source 1..20: mol_type = other DNA
tgttctcagggagatcttgg

SEQ ID NO: 8 - DNA, 20 bp, synthetic construct
  source 1..20: mol_type = other DNA
tttggaagcatccatcattt

SEQ ID NO: 9 - DNA, 20 bp, synthetic construct
  source 1..20: mol_type = other DNA
aagttggtcgagctgtatcg

SEQ ID NO: 10 - DNA, 20 bp, synthetic construct
  source 1..20: mol_type = other DNA
gagtttgatctggaggctca

SEQ ID NO: 11 - DNA, 20 bp, synthetic construct
  source 1..20: mol_type = other DNA
gcaaggaagattcgttcaga

SEQ ID NO: 12 - DNA, 20 bp, synthetic construct
  source 1..20: mol_type = other DNA
tcatctcactccaacgatca

SEQ ID NO: 13 - DNA, 20 bp, synthetic construct
  source 1..20: mol_type = other DNA
aacaaatgcaggaagctttg

SEQ ID NO: 14 - DNA, 20 bp, synthetic construct
  source 1..20: mol_type = other DNA
cctggtgtccaatagtctgg